(19) United States
(12) Patent Application Publication
(10) Pub. No.: US 2016/0303038 A1
(43) Pub. Date: Oct. 20, 2016

(54) ORAL FILMS

(71) Applicants: Harshal Ashok PAWAR, Maharashtra Kalyan (West) (IN); DINECT SARL, Maharashtra Thane (West) (IN)

(72) Inventors: Akanksha Bindeshwari Prasad YADAV, Maharashtra Thane (West) (IN); Harshal Ashok PAWAR, Maharashtra Kalyan (West) (IN); Amit Ashok CHIVATE, Maharashtra Mumbai (IN)

(21) Appl. No.: 15/038,451

(22) PCT Filed: Nov. 20, 2014
(86) PCT No.: PCT/IN2014/000727

§ 371 Date: May 21, 2016

(30) Foreign Application Priority Data
Nov. 21, 2013 (IN) 3658/MUM/2013

(51) Int. Cl. A61K 9/00; A61K 31/454; A61K 31/4545; A61K 31/451; A61K 31/551; A61K 31/522; A61K 8/02; A61Q 11/00; A61K 8/81; A61K 8/73; A61K 47/32; A61K 47/36; A61K 47/34; A61K 8/86; A61K 31/137; A61K 31/12; A61K 31/4418

(52) U.S. Cl. CPC A61K 9/006; A61K 31/4418; A61K 31/454; A61K 31/4545; A61K 31/451; A61K 31/551; A61K 31/522; A61K 8/0216; A61Q 11/00; A61K 8/8129; A61K 8/73; A61K 47/32; A61K 47/36; A61K 47/34; A61K 8/86; A61K 31/137; A61K 31/12; A61K 2800/92

(57) ABSTRACT

The invention relates to an oral film made from a copolymer of polyvinyl alcohol and polyethylene glycol and at least one gum. The oral films may further contain a medicated active and a non-medicated component. The film upon application, depending upon the quantity of gum and other additives may quickly disintegrate releasing its contents in the mouth or stay adhered for the period of greater than an hour, greater than two hours and preferably for around four hours or more.

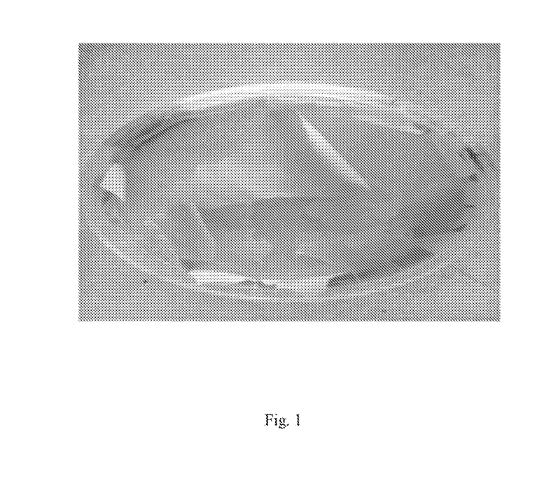

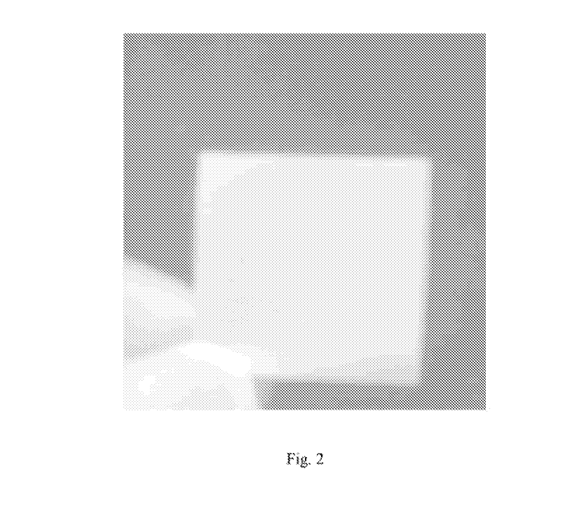

### CROSS REFERENCE TO RELATED APPLICATIONS

The present application is a National stage application from the PCT application PCT/IN2014/000727 filed Nov. 20, 2014, which claims priority to Indian application 3658/MUM/2013 filed Nov. 21, 2013.

### FIELD OF INVENTION

The present invention relates to an oral film made from a copolymer of polyvinyl alcohol and polyethylene glycol and at least one gum. The film may further contain a medicated active or a non-medicated active component. The non-medicated active component is selected from the group consisting of stimulants, flavors, mouth freshening agents, cooling agents, teeth whitening agents and food flavoring agents. The film optionally comprises of additional excipient such as additional film forming agents, plasticizers, surfactants, binders, thickeners and stabilizers, disintegrants, fillers, sweeteners, saliva stimulating agents, flavoring agents, mouth freshening/breath freshening agents, cooling agents, gelling agents, coloring agents and effervescence agents. Depending upon the quantity of gum and other additives, the film upon application may quickly disintegrate releasing its contents in the mouth.

### BACKGROUND OF THE INVENTION

Oral films have long been popular with patients as they are easy to consume by people of all ages. Therefore the compositions subject of the invention is the ideal solution for ambulatory treatment. They allow rapid administering of an active substance without having recourse to a liquid to aid swallowing. The films such as described in the invention can be placed directly in the oral cavity e.g. on the palate or under the tongue where they disintegrate almost immediately preferably within a minute and more preferably within few seconds.

Said property is therefore of particular advantage for the treatment of nausea, and travel sickness when there is a need for rapid administering of medicine and water for swallowing the pill might not be available. The compositions of the present invention also have an advantage for very young or elderly patients, who have trouble swallowing solid forms.

The patent WO0018835 disclosed film compositions comprising modified starches and setting system comprising gums.

The patent WO2012021710 disclosed film forming material comprises of a gum polysaccharide (50%-98%), one or more soluble fillers (1.5%-60%) texture altering ingredients.

The patent WO0191721 disclosed a film-forming composition, comprising: starch material having a dextrose equivalent less than about 1 and selected from the group consisting of modified starch and waxy starch; gum; and plasticizer. The composition comprises 25-75% starch material, 25-75% plasticizer, and 0.1-15% gum. All film compositions described above require many ingredients for formation of films and often required external plasticizers making such films hygroscopic. Thus it is desirable to have few ingredients in a film and yet film should incorporate many actives and should be stable, non-hygroscopic and easy to handle.

### OBJECT OF THE INVENTION

The present invention attains one of the following objectives.

First object of this invention is to provide a simplified formulation of rapid disintegrating edible film.

It is an object of the present invention to provide an oral film made up of copolymer of polyvinyl alcohol and polyethylene glycol and gum which quickly disintegrates and gives fast release of its contents, appropriate mechanical properties and good stability to film matrix at room temperature.

It is one object of the present invention to provide a non-hygroscopic film without the use of an external plasticizer as the addition of an external plasticizer makes the film hygroscopic and reduces the stability of the film.

Yet another object of the invention is to provide an orally consumable film which allows the medication to get absorbed buccally, sublingually according to its solubility and permeability characteristics.

It is one more object of the present invention to provide quick disintegrating oral films that release its contents even before patients can spit it out.

It is an object of the invention to provide for the process of preparation of an oral film using the solvent casting method.

### SUMMARY OF THE INVENTION

First aspect of the invention is to provide an oral film having a novel composition of copolymer of polyvinyl alcohol and polyethylene glycol and at least one gum.

Second aspect of the invention is to provide oral film without use of external plasticizer thus making it less hygroscopic, easy to handle and stable.

Third aspect of the present invention is to provide oral films which provide a film composition which is easy to prepare and provides for quick disintegration of the drug in the mouth.

Yet another aspect of the invention is to provide an oral medicated/non-medicated film comprising copolymer of polyvinyl alcohol and polyethylene glycol and a combination of various gums selected from the group consisting of Cassia tora, guar gum, xanthan gum and gum ghatti and combinations thereof.

One more aspect of the present invention is to provide quick disintegrating oral films that can't be spitted out easily. The films quickly release the contents in the oral cavity and depending on the permeation and absorption characteristics, drugs or non-drugs can be absorbed orally, buccally, sublingually.

One more aspect of present invention is to prepare bioadhesive films that adhere to tongue or oral mucosa for at least around one hour, preferably around two hours and more preferably around four hours or more. This can be achieved by carefully controlling gum concentration in the film and by adding gelling agent and or mucoadhesive polymers.

### BRIEF SUMMARY OF THE DRAWINGS

The drawing 1/2 attached hereto describes the Olanzapine oral film with one gum and lacking sufficient elasticity and tensile strength.

The drawing 2/2 attached hereto describes the Domperidone maleate oral film prepared according to the present invention described under example 2.

### DETAILED DESCRIPTION OF THE INVENTION

The present invention provides two types of oral films. First, quick disintegrating which disintegrate within couple of minutes, preferably in less than 1 minute and most preferably in less than 30 seconds. The other type of film is not meant for quick disintegration. Both types of films adhere to oral mucosa or tongue, however quick disintegrating films adhere for less than a minute whereas mucoadhesive films stay longer for more than 30 minutes, preferably more than an hour or even longer when such application is desired. Thus mucoadhesive films that can adhere for an hour, two hours, four hours and even longer can be prepared using present invention.

Both the films are made up of 1) copolymer of polyvinyl alcohol (PVA) and polyethylene glycol (PEG) and 2) at least one gum. For certain films, combination of two or more gums are required as illustrated by the examples. Particularly for insoluble drugs, combination of gums was found desirable, although further attempts are being made to make films of insoluble drugs with one gum. Gum is chosen from the group consisting of Cassia tora, xanthan gum, guar gum and gum ghatti. By carefully controlling gum concentration and adding further agents, one can make the film quickly disintegrating or mucoadhesive.

Copolymer of polyvinyl alcohol and polyethylene glycol has been commonly used in tablet coating particularly when moisture barrier coat is required for moisture sensitive drugs. However over several years no oral film preparation incorporating this copolymer has been commercially exploited. It is difficult to make oral films from this polymer and several attempts of preparing films have failed as they led to lump formation in the oral cavity. Present inventors also made an attempt, to prepare oralfilms of polyvinyl alcohol (62%) and polyethylene glycol (21%) in around 3:1 ratio. They added Cassia tora gum around 4% to such films. However those films were too thin to handle.

Thus several attempts to prepare oral films from combination of polyvinyl alcohol and polyethylene glycol and using copolymer of the two failed either because films were thin or because lump formation in oral cavity.

Present inventors have surprisingly found that adding at least one gum to the said Copolymer of polyvinyl alcohol and polyethylene glycol in certain proportion completely takes care of lump formation and provides smooth, aesthetic films which are stable, less hygroscopic and can incorporate several drug and non-drug components. Sometimes, combination of two or more gums is required. The gums act as stabilizers and film thickeners. Preferably, Copolymer of polyvinyl alcohol and polyethylene glycol is Kollicoat IR®, marketed by BASF. Its molecular weight is around 45000 Da and it is easily soluble in water. The two parts of copolymer of polyvinyl alcohol and polyethylene glycol contribute towards the mechanical properties of the film obtained. The PVA part imparts film-forming properties whilst the PEG part behaves as internal plasticizer. Hereinafter the said copolymer of polyvinyl alcohol and polyethylene glycol is also referred as copolymer.

The film of present invention has the said copolymer and at least one gum. Gum can be selected from the group consisting of Cassia tora, xanthan gum, guar gum and gum ghatti and combinations thereof. The Cassia tora gum is most preferred. Cassia tora gum can be micronized to get further better results. The particle size with sauter mean diameter from around 10 nm to 1000 nm is most preferred. The oral film made up of a copolymer of polyvinyl alcohol and polyethylene glycol and Cassia tora disintegrates within a minute and preferably within 30 seconds in mouth when placed on tongue.

Films of present invention comprise of 1) copolymer of polyvinyl alcohol and polyethylene glycol as film forming agent having internal plasticizer and 2) gum. Film forming agent can be used from around 40% to around 99% and gum is from around 0.5% to around 15%. Preferred gum concentration is from 1-10% and most preferred concentration is from 2-6%. Placebo films containing 99% copolymer and 1% gum are prepared as control for various testing experiments.

The preferred gum is Cassia tora gum obtained from Cassia tora L., (Cassia obtusifolia L.), Caesalpiniaceae, is a wild crop and grows in most parts of India as a weed. Cassia tora gum is a natural gelling agent which has industrial and food applications made commercially from the seed. Cassia grows in hot, wet, tropical climates both wild and commercially. Cassia is a tonic, carminative and stimulant. Cassia contains 1-2% volatile cassia oil, which is mainly responsible for the spicy aroma and taste. The primary chemical constituents of Cassia include cinnamaldehyde, gum, tannins, mannitol, coumarins, and essential oils (aldehydes, eugenol, and pinene); it also contains sugars, resins, and mucilage, among other constituents.

Cassia gum is the purified flour from the endosperm of the seeds of Cassia tora and Cassia obtusifolia which belong to the leguminosae family. The intended use of Cassia gum is as thickener, emulsifier, foam stabilizer, moisture retention agent and/or texturizing agent in cheese, frozen dairy desserts and mixes, meat products and poultry products.

Adhesion of the copolymer of polyvinyl alcohol and polyethylene glycol in human oral cavity mucosal membranes was improved by addition of gums, preferably Cassia tora gum making it almost impossible to spit because of instantaneous disintegration. Thus gums completely change the disintegration pattern of the copolymer which is by folding originally.

The combination of the copolymer of polyvinyl alcohol and polyethylene glycol and gum has been used in a ratio of 99:1 to 5:1, preferably between 25:1 to 8:1 or most preferably between 20:1 to 10:1. (differs for different drugs).

When drugs are incorporated into films of present invention, they are used from 0.5% to 60% w/w of film composition, preferably from 1% to 40% of the film composition. This allows one to incorporate large number of drugs with variable doses. For example, inventors have successfully prepared film of repaglinide (0.5 mg), amlodipine besylate (2.5 mg), domperidonemaleate (10 mg) olanzapine (20 mg) and of several other drugs.

Inventors have also successfully prepared stimulant as well as mouth/breath freshening films and flavoured films incorporating caffeine, menthol, thymol, flavours etc. which we collectively termed as non-medicated films. However, it is understood that the film can simultaneously contain drug and flavor, breath freshening agents and such films are also part of the present invention.

Thus films of present invention can contain drugs, food additives, nutraceutical bioactive agents, biological bioactive agents, breath freshening and mouth freshening agents, stimulants and likes and combinations thereof.

The drugs that can be suitably incorporated into the films of present invention can be selected from the group consisting of calcium channel blockers, such as amlodipine besylate, felodipine and the like; beta-blockers, such as metoprolol, bisoprolol, carvedilol; ACE inhibitors, such as benazepril, captopril, enalapril, lisinopril, moexipril, perindopril, quinapril, ramipril, trandolapril; vasodilators such as hydralazine, nicardipine, nitroglycerin; alpha-1 adrenergic blockers, such as alfuzosin, doxazosin, prazosin, tamsulosin, terazosin; anti-emetics, such as ondansetron, dolasetron, granisetron, aprepitant, metoclopramide, prochlorperazine, scopolamine; angiotensin II receptor blockers, such as candesartan, losartan, olmesartan, telmisartin and valsartan; anti-arrhythmics, such as atropine, dofetilide, moricizine, verapamil; narcotic analgesics, such as buprenorphine, naltrexone, suboxone, codeine, varenicline, and the like; aldosterone antagonists, such as eplerenone and spironolactone; alzheimer's disease medications, such as donepezil, galantamine, rivastigmine, tacrine and memantine; anti-epileptics, such as clonazepam, diazepam, lamotrigine phenytoin, pregabalin, primidone, tiagabine, topiramate, zonisamide; anti-migraines, such as almotriptan, eletriptan, frovatriptan, naratriptan, rizatriptan, sumatriptan and zolmitriptan; antimicrobial agents, such as triclosan, cetylpyridiumchloride, domiphen bromide, and the like; anti-tussives, such as dextromethorphan hydrobromide, menthol and the like; decongestants, such as pseudoephedrine hydrochloride, pseudoephedrine sulphate, phenylephrine, phenylpropanolamine, and the like; expectorants, such as guaifenesin, ipecac, and the like; anti-diarrheals, such as loperamide and the like; H₁ receptor antagonists, such as desloratidine, loratidine, famotidine, fexofenadine, levocetrizine, mirtazapine, and the like; anti-diabetic agents, such as acarbose, miglitol, rosiglitazone, osiglitazone, repaglinide, glimepiride, glyburide, glipizide; antihistamines, such as brompheniramine maleate, chlorpheniramine maleate and the like; neuroleptics, such as asenapine, aripiprazole, chlorpromazine, clozapine, fluphenazine, haloperidol, loxapine, molindone, olanzapine, perphenazine, pimozide, quetiapine, risperidone, thioridazine, trifluoperazine, ziprasidone; antihistamines, such as cetirizine, desloratadine, fexofenadine, loratadine, chlorpheniramine, clemastine, cyprobeptadine, diphenhydramine, hydroxyzine and promethazine; antispasmotics, such as dicyclomine, propantheline, simethicone, hyoscyamine; benzodiazepines and non-benzodiazepine sedatives, such as alprazolam, buspirone, chlordiazepoxide, chlorazepate, clonazepam, diazepam, estazolam, eszopiclone, flurazepam, lorazepam, oxazepam, ramelteon, temazepam, triazolam, zaleplon and zolpidem; beta blockers, such as atenolol, betaxolol, bisoprolol, carvedilol, esmolol, labetalol, metoprolol, propranolol; benign prostatic hypertrophy medications, such as doxazosin, dutasteride, finasteride, tamsulosin and terazosin; corticosteroids, such as budesonide, cortisone acetate, dexamethasone fludrocortisones, hydrocortisone, methylprednisolone and prednisone; erectile dysfunction agents, such as sildenafil, tadalafil, vardenatil; H₂ receptor blockers, such as famotidine; dry mouth syndrome, such as pilocarpine; nitrates, such as isosorbidedinitrate, isosorbidemononitate, nitroglycerin; parkinson's disease treatments, such as amantadine, benztropine, bromocriptine, pergolide, pramipexole, ropinirole, selegiline, and trihexyphenidyl; Opioid analgesics, such as fentanyl, hydromorphone and morphine; renal failure medications, such as, calcitriol, doxercalciferol, paricalcitol; pulmonary medications, such as, albuterol, metaproterenol, budesonide, montelukast, zafirlukast; anti-spasmodic, such as baclofen; immuno suppressants, such as prednisolone sodiumphosphate; statins, such as atorvastatin, fluvastatin, lovastatin, pravastatin, rosuvastatin, simvastatin, and ezetimibe; proton pump inhibitors, such as, lansoprazole, loperamide; stimulants, such as atomoxetine, benzphetamine, caffeine, nicotine, phentermine hydrochloride, dexmethylphenidate, dextroamphetamine, diethylpropion, methylphenidate, armodafinil, pemoline, phentermine and sibutramine; anti-thyroid agents such as methimazole and propylthiouraciland the salts, esters, enantiomers of the above and the likes and combinations thereof.

Food or nutraceutical bioactive agents include, but are not limited to, nootropies such as vinpocetine, Vitamins, such as vit. D, Vit. B and the likes, constituents in foods or dietary supplements that are responsible for changes in health status, such as components of plants, especially fruits and vegetables, e.g., soy which contains isoflavones and phytoestrogens, tomatoes which contain lycopene, potential weight loss agents and energy boosters such as octopamine hydrochloride.

It is to be understood that the present invention is a platform technology that can be used for any medicine or non-medicinal active. For very high daily doses such as 500 mg-2 gm, the bigger films can be prepared having more surface area using the same invention. Inventors have successfully prepared very big placebo films and incorporation of high dose is possible. For very high daily doses, the bigger films or frequent administration can be adopted as against once a day administration and smaller films. Hence invention should not be construed as restricted to a particular medicine or non-medicinal active component and it should not be construed as restricted to a particular dose range. The spirit of invention is to include any medicine or non-medicinal active and such addition with few variations is within the scope of the invention.

According to nature of drug/non drug component to be incorporated into film, several additional excipients additives are added into the film such as additional film forming agents, plasticizers, surfactants, binders, thickeners and stabilizers, disintegrants, fillers, sweeteners, saliva stimulating agents, flavouring agents, mouth freshening/breath freshening agents, cooling agents, gelling agents, colouring agents and effervescence agents. Additionally, when the films are mucoadhesive, they contain mucoadhesive agents incorporated into the films.

Additional Film forming agents selected from the group consisting of Pullulan, Hydroxypropylmethyl cellulose, Hydroxyethyl cellulose, Hydroxypropyl cellulose, Polyvinyl pyrrolidone, Polyvinyl alcohol, Carboxymethyl cellulose, Polyethylene oxide, Sodium alginate, tragacanth gum, guar gum, gum ghatti, veegum, locust bean gum, karava gum, a copolymer of polyvinyl alcohol and polyethylene glycol, acacia gum, arabic gum, polyacrylic acid, methylmethacrylate copolymers, carboxyvinyl polymers, amylose, high amylose starch, hydroxypropylated high amylose starch, dextrin, pectin chitin, chitosan, levan, elsinan, collagen, gelatin, zein, gluten, soy protein isolate, whey protein isolate, casein carrageenan, starch, natural gums and combinations thereof and edible natural plant extract such as konjac and pectin.

Plasticizers render film forming agents more useful by altering the physical properties of the films imparting flexibility, softness by reducing the glass transition temperature (Tg) of the polymer and thereby enhancing the mobility of the polymeric chain. And can range from approximately 0% to 20% of the dry film weight. Some of the examples include but are not limited to glycerol, propylene glycol, lowmolecular weight polyethylene glycols, phthalate derivatives like dimethyl, diethyl, castor oil and dibutyl phthalate, citrate derivatives such as tributyl, triethyl, acetyl citrate, triacetin and glycerol monoesters with fatty acids or other pharmaceutical polyalcohols. The concentration of polyalcohols can range between 0.1 and 5%.

Casein, pullulan, starchare the useful binding agents, can be used in amounts ranging from 0 to 20%.

Suitable thickening agents include but are not limited to natural gums like xanthan gum, locust bean gum, guar gum, carrageenan, tragacanth gum, gum arabic, acacia gum, sodium alginate, polyacrylates such as carboxyvinyl copolymers, carbomers, and cellulosic derivatives such as hydroxypropyl methyl cellulose, hydroxyethyl cellulose, hydroxypropyl cellulose, used alone or in combinations in amounts ranging from 0 to 10%.

Filler can be added to reduce the slimy texture of the film composition. Examples include but are not limited to magnesium carbonate, calcium carbonate, calcium phosphate, calcium sulphate, magnesium silicate, aluminium silicate, ground lime stone, clay, talc, titanium dioxide, microcrystalline cellulose, and can range from approximately 10% to 90% of the dry film weight.

Sucrose, dextrose, fructose, maltose, sorbitol. First generation artificial sweeteners such as Saccharin, cyclamate, aspartame. Second generation sweeteners such as Acesulfame-K, Sucralose, alitame, neotame. Natural sweeteners such as rebiana, stevia, liquorice extract.

Saliva stimulating agents are used alone or in combination between 2 to 6% weight of the film. Examples include glucose, fructose, xylose, maltose, lactose, sucrose, aspartame, sodium saccharin, fruit acids, such as citric acid, malic acid, tartaric acid.

Natural and artificial flavours can be used and selected from synthetic flavour oils, flavouring aromatics and or oils, oleo resins and extracts from different part of the plant like leaves, flower, fruit, bark, seeds. Some of examples include but are not limited to fruit flavours, such as apple, raspberry, cherry, strawberry, pineapple; oils, such as peppermint, cinnamon, spearmint, nutmeg; fruit flavours, such as vanilla, cocoa, coffee, chocolate, citrus; fruit essence, such as apple, raspberry, cherry, pineapple. Cooling agents, such as monomethyl succinate, WS3, WS23, ultracoll II, thymol. Coloring agents, such as Titanium dioxide, FD & C approved coloring agent can be used.

Effervescence agents can be optional for improved compliance with paediatrics and effective coating of the oral cavity with flavours and be achieved by including small bits of gas releasing agent in the formulation.

Gums other than Cassia tora can also be incorporated. Cassia tora provides best disintegrating film with less disintegration time. However other gums can also provide films that disintegrate within a minute. Corn starch can be further added to improve film properties. It also helps in achieving desired disintegration. Corn starch swells in the mouth on consumption of the film by absorbing saliva and decreases the disintegration time for the film.

Cassia tora also can be used in combination with other gums. Similarly, combination of various gums can be used to provide sufficient viscosity as well as desired disintegration.

The film may include one or more of the following additional excipients including but not limited to surfactants/wetting agents, preservatives, antioxidants, antimicrobial agents, emulsifying agents, fragrances, pH modifiers, permeation enhancers, solubilizers, sulphur precipitating agents, polyethylene oxides. Surfactants such as Poloxamer, sodium lauryl sulphate, polysorbates, polyoxyethylene alkyl ethers (Brij), and polyoxyethylenesorbitanfattyacidesters and the like can be used. Preservatives such as propyl paraben, methyl paraben and the like can be used. Antioxidants such as beta hydroxyl toluene, propyl gallate, butyl hydroxyanisole and the like can be used. Antimicrobials such as EDTA, cetylperidium chloride, triclosan, quaternary ammonium compounds and the like can be used. Emulsifying agents such as triethanolamine stearate, veegum, acacia and the like can be used. pH modifiers such as citric acid, Sodium Bicarbonate, and the like can be used. Permeation enhancers such as sodium glycocholate and the like can be used. Solubilizers such as proplene glycols, polyethylene glycols and the like can be used. Alcoholic solvents such as ethanol and isopropyl alcohols can also be used to solubilize actives.

The film according to the present invention is suitable for oral, mucosal or sublingual administration of any medicinal or non-medicinal active agent. It is the nature of an active agent which would play role in the way it will be absorbed. Hence the films of the present invention even provides opportunities to include several drugs which otherwise are not administered orally due to stability, first pass metabolism or for any other reasons.

Film properties of the film of the present invention are normally as follows, however films of properties other than these can also be prepared.
1) Film weight from around 10 mg to around 200 mg.
2) Disintegration time—within 2-3 minutes, preferably in less than a minute
3) Dissolution of active—more than 80% release in 15 minutes, preferably in less than 10 minutes.
4) Content uniformity—satisfactory and within range
5) Thickness—0.05 mm to 1 mm
6) Surface area 0.5 cm² to 15 cm²
7) pH 5-8, preferably 6-7 unless highly acidic/basic active agents are used.

The process used for preparing films of present invention includes solvent casting methods as follows and comprises of the following steps
1) Adding the film forming agent to 50% of the purified water under continuous stirring;
2) Adding a gum to above prepared mixture;
3) Adding a sweetener and stirring until complete solubilization;
4) The active agent is either added to this mixture or in cases where the agent is insoluble, a solution of the same is made with a solubilizing agent (surfactant) which is then added to the above mixture.
5) Casting said mixture on a casting surface and dried for more than 15 hrs.
6) The prepared film is cut in desired dimension, packed manually in foil, sealed and kept in air tight container.

The invention is illustrated below by the following non-limiting examples.

### EXAMPLES

Suitable batch sizes for all films below were from about 20 films-about 50 films. For 20 films, amount of water required was around 10 ml. This quantity of water was divided into two parts, each of 5 ml.

### Example 1

Process of Preparing Amlodipine Besylate (23 mg) Oral Films

In 50% quantity of purified water, hydrophilic polymer (A copolymer of polyvinyl alcohol and polyethylene glycol) was added under constant stirring using a digital magnetic stirrer until dissolved followed by addition of Cassia tora gum under constant stirring for about one hour. Sweetener (aspartame) was added and stirred until complete solubilisation. Amlodipine besylate was incorporated in the film forming mixture and constantly warmed to about 40 to 45° C. followed by addition of remaining portion of purified water and stirred under agitation. The mixture was then casted on polypropylene petri dish and dried using hot air oven at 45-50° C. for 24 hrs. The prepared film was cut in desired dimension, packed manually in foils, sealed and kept in air tight container till further evaluation for quality parameters.

TABLE 1
Composition of oral film of Amlodipine besylate

| Ingredients | mg/film | % w/w | mg/film | % w/w |
|---|---|---|---|---|
| Amlodipine besylate | 2.50 | 8.06 | 10 mg | 25.96 |
| A copolymer of | 25.92 | 83.58 | 25.92 | 67.30 |
| polyvinyl alcohol and | | | | |
| polyethylene glycol | | | | |
| Cassia toragum | 1.49 | 4.82 | 1.49 | 3.88 |
| Aspartame | 1.09 | 3.53 | 1.09 | 2.84 |
| Purified water | — | — | — | — |
| Total | 31.01 | 100 | 38.55 | 100 |

Film Properties

| Disintegration time | 1.49 | 15 |
|---|---|---|
| (sec); n = 3 | | |
| Dissolution time | 270 | 30 |
| (Time required for | | |
| >80% drug release in | | |
| seconds) | | |
| Uniformity of drug | 101.07 ± 3.38 | 98.08 ± 1.81 |
| content, n = 3 | | |
| Thickness (mm) | 0.12 ± 0.018 | 0.1846 ± 0.025 |
| n = 5 | | |

Note:
% w/w and mg/film assuming all solvent evaporated

### Example 2

Process of Preparing Domperidone Maleate (10 mg) Oral Films

In 50% quantity of purified water, hydrophilic polymer (A copolymer of polyvinyl alcohol and polyethylene glycol) was added under constant stirring using a digital magnetic stirrer until dissolved followed by addition of Cassia tora gum under constant stirring for about one hour. Sweetener (aspartame) was added and stirred until complete solubilization. In remaining portion of purified water, poloxamer was dissolved followed by addition of Domperidone maleate under constant mixing. The drug dispersion as added to polymer phase under constant stirring. The mixture as then casted on polypropylene petri dish and dried using hot air oven at 45-50° C. for 24 hrs. The prepared film was cut in desired dimension, packed manually in foil, sealed and kept in air tight container till further evaluation for quality parameters.

TABLE 2
Composition of oral film of Domperidone maleate (10 mg)

| Ingredients | mg/film | % w/w |
|---|---|---|
| Domperidone maleate | 10.00 | 21.77 |
| A copolymer of polyvinyl alcohol | 29.90 | 65.09 |
| and polyethylene glycol | | |
| Cassia toragum | 1.89 | 4.12 |
| Aspartame | 1.09 | 2.38 |
| Poloxamer 188 | 3.04 | 6.61 |
| Purified water | — | — |
| Total | 45.93 | 100 |

| Film Properties | |
|---|---|
| Disintegration time (sec), n = 3 | 10.25 |
| Dissolution time | 330 |
| (Time required for >80% drug | |
| release in seconds) | |
| Uniformity of drug content, n = 3 | 97.31 ± 0.97 |
| Thickness (mm), n = 5 | 0.1670 ± 0.034 |

Note:
% w/w and mg/film assuming all solvent evaporated

### Example 3

Process of Preparing Loratidine (10 mg) Oral Films

Process is same as in Example 2, except that mint flavor is added after dissolving poloxamer.

TABLE 3
Composition of Loratidine(10 mg) oral film.

| Ingredients | mg/film | % w/w |
|---|---|---|
| Loratidine | 10.00 | 22 .65 |
| A copolymer of polyvinyl alcohol | 27.91 | 63.23 |
| and polyethylene glycol | | |
| Cassia toragum | 1.79 | 4.06 |
| Aspartame | 1.09 | 2.46 |
| Poloxamer 188 | 3.04 | 6.88 |
| Flavour | 0.29 | 6.77 |
| Purified water | — | — |
| Total | 44.14 | 100 |

| Film Properties | |
|---|---|
| Disintegration time (sec), n = 3 | 6.04 |
| Dissolution time (Time required | 300 |
| for 100% drug release in seconds) | |
| Uniformity of drug content, n = 3 | 95.68 ± 0.59 |
| Thickness (mm), n = 5 | 0.1713 ± 0.025 |

Note:
% w/w and mg/film assuming all solvent evaporated

### Example 4

Process of Preparing Repaglinide (0.5 mg) Oral Films

Process of preparing Repaglinide (0.5 mg) oral films is same as in Example 2.

TABLE 4
Composition of Repaglinide oral film

| Ingredients | mg/film | % w/w |
|---|---|---|
| Repaglinide | 0.50 | 1.68 |
| A copolymer of polyvinyl | 25.42 | 85.85 |
| alcohol and polyethylene | | |
| glycol | | |
| Cassia toragum | 1.39 | 4.71 |
| Aspartame | 0.99 | 3.36 |
| Poloxamer 188 | 1.29 | 4.37 |
| Purified water | — | — |
| Total | 29.61 | 100 |

| Film Properties | |
|---|---|
| Disintegration time (sec) | 10.98 |
| n = 3 | |
| Dissolution time | 60 |
| (Time required for 100% | |
| drug release in seconds) | |
| Uniformity of drug content, | 96.07 ± 0.88 |
| n= 3 | |
| Thickness (mm), n = 5 | 0.130 ± 0.012 |

Note:
% w/w and mg/film assuming all solvent evaporated

### Example 5

Olanzapine is highly insoluble drug unlike other drugs and films required combination of two gums and films of one gum could not be prepared as they lacked elasticity and tensile strength.

Process of Preparing Olanzapine Oral Films

In 50% quantity of purified water, hydrophilic polymer (A copolymer of polyvinyl alcohol and polyethylene glycol) was added under constant stirring using a digital magnetic stirrer until dissolved followed by addition of gums and stirred for about 30 min. In remaining portion of purified water poloxamer was dissolved followed by the drug, stirred, sweetener, flavour, starch was incorporated under constant mixing. The drug dispersion was added to polymer phase under constant stirring. The mixture was then casted on polypropylene petri dish and dried using hot air oven at 45-50° C. for 24 hrs. The prepared film was cut in desired dimension, packed manually in foil, sealed and kept in air tight container till further evaluation for quality parameters.

TABLE 5
Composition of Olanzapine oral film,

| Ingredients | % w/w | mg/film |
|---|---|---|
| Olanzapine | 39.75 | 20 |
| A copolymer of polyvinyl alcohol and | 47.56 | 23.92 |
| polyethylene glycol | | |
| Xanthan gum | 0.69 | 0.34 |
| Guar gum | 0.89 | 0.44 |
| Corn starch | 1.38 | 0.69 |
| Poloxamer 188 | 3.46 | 1.74 |
| Aspartame | 2.47 | 1.24 |
| Vanilla flavour | 3.76 | 1.89 |
| Purified water | — | — |
| Total | 100 | 50.30 |

Film Properties

| | |
|---|---|
| Disintegration time (see), n = 3 | 12.63 |
| Dissolution time (Time required for 100% drug | — |
| release in seconds) | |
| Uniformity of drug content | 96.9 ± 2.605 |
| Thickness (mm), n = 5 | 0.187 ± 0.017 |

Note:
% w/w and mg/film assuming all solvent evaporated

### Example 6

Process of Preparing Olanzapine Oral Film

In 50% quantity of purified water, hydrophilic polymer (A copolymer of polyvinyl alcohol and polyethylene glycol) was added under constant stirring using a digital magnetic stirrer until dissolved followed by addition of Cassia tora gum and stirred for about 30 min. Xanthan gum was added and stirred till homogenous mixture formed. In remaining portion of purified water poloxamer was dissolved followed by the drug, stirred, sweetener was added under constant mixing. The drug dispersion was added to polymer phase under constant stirring. The mixture was then casted on polypropylene petri dish and dried using hot air oven at 45-50° C. for 24 hrs. The prepared film was cut in desired dimension, packed manually in foil, sealed and kept in air tight container till further evaluation for quality parameters.

TABLE 6
Composition of Olanzapine oral film.

| Ingredients | % w/w | mg/film |
|---|---|---|
| Olanzapine | 39.79 | 20 |
| A copolymer of polyvinyl alcohol and | 49.59 | 24.92 |
| polyethylene glycol | | |
| Cassia toragum | 3.17 | 1.59 |
| Xanthan gum | 0.69 | 0.34 |
| Poloxamer 188 | 4.46 | 2.24 |
| Aspartame | 2.28 | 1.14 |
| Purified water | — | — |
| Total | 100 | 50.25 |

Film Properties

| | |
|---|---|
| Disintegration time (sec), n = 3 | 10.23 |
| Dissolution time (Time required for 100% drug | 360 |
| release in seconds) | |
| Uniformity of drug content | 100.5 ± 3.97 |
| Thickness (mm); n = 5 | 0.181 ± 0.003 |

Note:
% w/w and mg/film assuming all solvent evaporated

### Example 7

Process of Preparing Caffeine (15 mg) Oral Films

In 50% quantity of purified water, hydrophilic polymer (A copolymer of polyvinyl alcohol and polyethylene glycol) was added under constant stifling using a digital magnetic stirrer until dissolved followed by addition of gums and stirred for about 30 min. In remaining portion of purified water poloxamer was dissolved followed by caffeine, stirred, sweetener, flavour, starch was incorporated under constant mixing. The drug dispersion was added to polymer phase under constant stirring. The mixture was then casted on polypropylene petri dish and dried using hot air oven at 45-50° C. for approx. 24 hrs. The prepared film was cut in desired dimension, packed manually in foil, sealed and kept in air tight container till further evaluation for quality parameters.

TABLE 7
Composition of Caffeine oral film.

| Ingredients | % w/w | mg/film |
|---|---|---|
| Caffeine | 8.15 | 2.5 mg |
| A copolymer of polyvinyl alcohol and | 81.28 | 24.92 |
| polyethylene glycol | | |
| Gum ghatti | 1.62 | 0.49 |
| Corn starch | 3.19 | 0.69 |
| Poloxamer 188 | 1.62 | 0.49 |
| Aspartame | 3.57 | 1.09 |
| Chocolate flavour | 1.46 | 0.44 |
| Purified water | — | — |
| Total | 100 | 28.16 |

Film Properties

| | |
|---|---|
| Disintegration time (sec), n = 3 | 11.96 |
| Dissolution time(Time required for 100% drug | — |
| release in seconds) | |
| Uniformity of drug content | — |
| Thickness (mm), n = 5 | 0.135 ± 0.011 |

Note:
% w/w and mg/film assuming all solvent evaporated

### Example 8

Process of Preparing Caffeine Oral Film

In 50% quantity of purified water, hydrophilic polymer (A copolymer of polyvinyl alcohol and polyethylene glycol) was added under constant stirring using a digital magnetic stirrer until dissolved followed by addition of Cassia tora gum and stirred for about 30 min. Xanthan gum was added and stirred till homogenous mixture formed. In remaining portion of purified water poloxamer was dissolved followed by the caffeine, stirred, sweetener was added under constant mixing. The Caffeine dispersion was added to polymer phase under constant stirring. The mixture was then casted on polypropylene petri dish and dried using hot air oven at 45-50° C. for approx. 24 hrs. The prepared film was cut in desired dimension, packed manually in foil, sealed and kept in air tight container till further evaluation for quality parameters.

TABLE 8
Composition of Caffeine oral film.

| Ingredients | % w/w | mg/film | % w/w | mg/film |
|---|---|---|---|---|
| Caffeine | 27.66 | 10 mg | 835 | 2.5 mg |
| A copolymer of polyvinyl alcohol | 66.39 | 23.92 | 83.31 | 24.92 |
| and polyethylene glycol | | | | |
| Cassia toragum | 2.21 | 0.79 | 4.66 | 1.39 |
| Xanthan gum | 0.96 | 0.34 | — | — |
| Aspartame | 2.76 | 0.99 | 3.66 | 1.09 |
| Purified water | — | — | — | — |
| Total | 100 | 36.03 | 100 | 29.91 |

Film Properties

| | | |
|---|---|---|
| Disintegration time (sec), n = 3 | 10.95 | 5.89 |
| Dissolution time (Time required | — | — |
| for 100% drug release in seconds) | | |
| Uniformity of drug content | — | — |
| Thickness (min), n = 5 | 0.124 ± 0.019 | 0.121 ± 0.010 |

Note:
% w/w and mg/film assuming all solvent evaporated

### Example 9

Process of Preparation of Flavoured oral Films:

In 50% quantity of purified water, hydrophilic polymer (A copolymer of polyvinyl alcohol and polyethylene glycol) was added under constant stirring using a digital magnetic stirrer until dissolved followed by addition of gums and stirred for about 30 min. In remaining portion of purified water poloxamer was dissolved followed by the drug, stirred, sweetener, flavour, starch was incorporated under constant mixing. The flavour dispersion was added to polymer phase under constant stirring. The mixture was then casted on polypropylene petri dish and dried using hot air oven at 45-50° C. for approx. 24 hrs. The prepared film was cut in desired dimension, packed manually in foil, sealed and kept in air tight container till further evaluation for quality parameters.

TABLE 9
Composition of Flavoured oral film.

| Ingredients | % w/w | mg/film |
|---|---|---|
| A copolymer of polyvinyl alcohol and | 78.68 | 23.92 |
| polyethylene glycol | | |
| Xanthan gum | 1.14 | 0.348 |
| Guar gum | 1.14 | 0.34 |
| Corn starch | 2.62 | 0.79 |
| Poloxamer 188 | 1.63 | 0.49 |
| Aspartame | 3.27 | 0.99 |
| Rose flavour | 6.55 | 1.99 |
| Mint | 4.91 | 1.49 |
| Purified water | — | — |
| Total | 100 | 30.40 |

Film Properties

| | |
|---|---|
| Disintegration time (sec), n = 3 | 6.48 |
| Dissolution time (Time required for 100% drug | — |
| release in seconds | |
| Uniformity of drug content | — |
| Thickness (mm), n = 5 | 0.138 ± 0.032 |

Note:
% w/w and mg/film assuming all solvent evaporated

Example 10

Process of Preparing Breath Freshening Oral Film:

In 50% quantity of purified water, hydrophilic polymer (A copolymer of polyvinyl alcohol and polyethylene glycol) was added under constant stirring using a digital magnetic stirrer until dissolved followed by addition of Cassia tora gum and stirred for about 30 min. In remaining portion of purified water, menthol and thymol and sweetener was incorporated under constant mixing and under constant heating at a temperature of 40° C. The menthol and thymol containing dispersion was added to polymer phase under constant stirring. The mixture was then casted on polypropylene petri dish and dried using hot air oven at 45-50° C. for approx. 24 hrs. The prepared film was cut in desired dimension, packed manually in foil, sealed and kept in air tight container till further evaluation for quality parameters.

TABLE 10
Composition of breath freshening oral film.

| Ingredients | mg/film | % w/w |
|---|---|---|
| A copolymer of polyvinyl alcohol and | 28.91 | 76.92 |
| polyethylene glycol | | |
| Cassia tora gum | 1.39 | 3.71 |
| Aspartame | 1.09 | 2.91 |
| Menthol | 4.38 | 11.67 |
| Thymol | 1.79 | 4.77 |
| Purified water | — | — |
| Total | 37.58 | 100 |

Film Properties

| | |
|---|---|
| Disintegration time (sec), n = 3 | 6.89 |

Note:
% w/w and mg/film assuming all solvent evaporated
All films are tested for visual observation, pH and weight at initial time point and on stability found to be stable.

### Example 11

Process: The film forming polymer phase was prepared by dissolving A copolymer of polyvinyl alcohol and polyethylene glycol in 60% quantity of distilled water followed by addition of xanthan gum, locust, bean gum, guar gum and Cassia tora gum slowly under constant mixing for two hours. Flavoring agent, sweetener and starch were added under continuous stirring until dissolved. Phenylephrine HCl was slowly added to the polymer phase along with remaining portion of water and mixed uniformly to yield the final mixture which was then casted on polypropylene petri dish, dried in hot air oven and cut into units of 3.24 cm² and packed in individual pouches.

TABLE 11
Composition of oral quick disintegrating film of Phenylephrine HCl
(12.2 mg)

| | | % w/w |
|---|---|---|
| Ingredients | mg/film * | Dry film * |
| Phenylephrine | 12.21 | 23.62 |
| HCl | | |
| A copolymer of | 33.89 | 64.57 |
| polyvinyl | | |
| alcohol and | | |
| polyethylene | | |
| glycol | | |
| Xanthan Gum | 0.19 | 0.38 |
| Locust Bean | 0.64 | 1.15 |
| gum | | |
| Guar gum | 0.74 | 1.44 |
| Aspartame | 1.14 | 2.21 |
| Vanilla flavor | 0.99 | 1.91 |
| Corn starch | 0.69 | 1.33 |
| Cassia toragum | 1.14 | 2.21 |
| Distilled water | — | — |
| Total | 52.28 | 100.01 |

Film Properties

| | |
|---|---|
| Average Disintegration time | 2.39 ± 0.93 |
| (sec) | |
| Average Thickness (mm) | 0.153 ± 0.02 |
| Average Dissolution time | 150 |
| (Time required for >80% drug | |
| release in seconds) | |
| Uniformity of drug content | 98 ± 1.32 |

Note:
* Assuming all solvent evaporated

### Example 12

Process: The film forming polymer phase was prepared by dissolving A copolymer of polyvinyl alcohol and polyethylene glycol in 60% quantity of distilled water followed by addition of xanthan gum, locust bean gum, guar gum under constant stilling for about two hours. The drug phase was prepared by dissolving poloxamer in remaining portion of distilled water followed by addition of curcumin under constant mixing. Flavoring agents and sweetener were added to the polymer phase. The drug phase as slowly added to the polymer phase and mixed uniformly to yield the final homogenous mixture which was then casted on polypropylene petri dish, dried in hot air oven and cut into units of 4.5 cm² and packed in individual pouches.

TABLE 12
Composition of oral quick disintegrating film of Curcumin (36 mg)

| | | % w/w |
|---|---|---|
| Ingredients | mg/film * | Dry film * |
| Curcumin | 36 | 41.63 |
| A copolymer of | 42.92 | 49.63 |
| polyvinyl alcohol | | |
| and polyethylene | | |
| glycol | | |
| Xanthan Gum | 0.62 | 0.72 |
| Locust Bean gum | 0.9 | 1.04 |
| Guar gum | 0.83 | 0.96 |
| Poloxamer188 | 0.9 | 1.04 |
| Aspartame | 1.52 | 1.76 |
| Raspberyflavor | 1.38 | 1.60 |
| Mint flavor | 1.38 | 1.60 |
| Distilled water | — | — |
| Total | 86.46 | 100 |

Film Properties

| | |
|---|---|
| Average Disintegration time | 34.75 ± 0.95 |
| (sec) | |
| Average Thickness (mm) | 0.204 ± 0.01 |

Note:
* Assuming all solvent evaporated

### Example 13

Process: Same as described in Example 2. Oral quick disintegrating films prepared from a copolymer of polyvinyl alcohol and polyethylene glycol, Xanthan Gum, Locust Bean gum and Cassia tora gum. The prepared films were cut into units of 4.5 cm² and packed in individual pouches.

TABLE 13
Composition of oral quick disintegrating films containing a copolymer
of polyvinyl alcohol and polyethylene glycol, Xanthan Gum,
Locust Bean gum and Cassia tora gum.

| | mg/film* | mg/film* | mg/film* |
|---|---|---|---|
| | (% w/w | (% w/w | (% w/w |
| Ingredients | dry film*) | dry film*) | dry film*) |
| Venlafaxine | — | — | 25.33 |
| hydrochloride | | | (31.28) |
| Propranolol HCl | — | 10.03 | — |
| | | (15.75) | |
| Losartan Potassium | 12.44 | — | — |
| | (20.46) | | |
| A copolymer of | 42.30 | 47.07 | 48.46 |
| polyvinyl alcohol and | (69.57) | (73.90) | (59.82) |
| polyethylene glycol | | | |
| Xanthan Gum | 0.24 | 0.27 | 0.27 |
| | (0.40) | (0.43) | (0.34) |
| Locust Bean gum | 0.80 | 0.96 | 0.83 |
| | (1.32) | (1.52) | (1.02) |
| Cassia toragum | 1.439 | 1.59 | 1.59 |
| | (2.35) | (2.49) | (1.96) |
| Aspartame | 1.43 | 1.59 | 1.52 |
| | (2.35) | (2.49) | (1.88) |
| Raspberry flavor | 1.36 | 1.31 | — |
| | (2.2) | (2.06) | |
| Vanilla flavor | — | — | 1.31 |
| | | | (1.62) |
| Mint flavor | — | — | 0.83 |
| | | | (1.02) |
| Poloxamer 188 | 0.87 | 0.9 | 0.83 |
| | (1.4) | (1.41) | (1.02) |
| Distilled water | — | — | — |
| Total | 60.89 | 63.7 | 81 |
| | (100) | (100) | (100) |

Film Properties

| | | | |
|---|---|---|---|
| Average Disintegration | 6.56 ± 0.17 | 6.36 ± 0.06 | 28.93 ± 0.85 |
| time (sec) | | | |
| Average Thickness | 0.109 ± 0.012 | 0.115 ± 0.013 | 0.135 ± 0.016 |
| (mm) | | | |
| Average Dissolution | 80 | 270 | — |
| time | | | |
| (Time required for >80% | | | |
| drug release in | | | |
| seconds) | | | |
| Uniformity of drug | — | 100.35 ± 0.64 | — |
| content | | | |

Note:
*Assuming all solvent evaporated

### Example 14

Oral films prepared from a copolymer of polyvinyl alcohol and poly-ethylene glycol, Cassia tora gum and guar gum. Method for preparation of Eletriptan Hydrobromide is same as described in Example 2. Poloxamer was added as a wetting agent in preparation of Eletriptan HBr films. Process for preparation of Ephedrine HCl films is same as described in example 1. The prepared films were cut into units of 3.24 cm² and packed in individual pouches.

TABLE 14
Composition of oral quick disintegrating
films containing a copolymer of polyvinyl alcohol and
polyethylene glycol, Cassia tora gum and guar gum

| | | % w/w | | % w/w |
|---|---|---|---|---|
| | | Dry | | Dry |
| Ingredients | mg/film* | film* | mg/film* | film* |
| Ephedrine HCl | — | — | 12.51 | 24.27 |
| EletriptanHBr | 24.22 | 39.38 | — | — |
| A copolymer of polyvinyl | 32.4 | 51.67 | 34.89 | 67.69 |
| alcohol and polyethyene | | | | |
| glycol | | | | |
| Cassia toragum | 0.99 | 1.62 | 1.14 | 2.22 |
| Guar gum | 1.09 | 1.78 | 0.84 | 1.64 |
| Poloxamer188 | 0.49 | 0.81 | — | — |
| Aspartame | 1.19 | 1.94 | 1.09 | 2.12 |
| Vanilla mint flavor | 1.09 | 1.78 | 1.04 | 2.03 |
| Distilled water | — | — | — | — |
| Total | 61.51 | 100 | 51.54 | 100.00 |

Film Properties

| | | |
|---|---|---|
| Average Disintegration | 24.25 ± 1.5 | 18.11 ± 0.77 |
| time (sec) | | |
| Average Thickness (mm) | 0.142 ± 0.028 | 0.198 ± 0.014 |

Note:
*Assuming all solvent evaporated

### Example 15

Oral films prepared using a copolymer of polyvinyl alcohol and polyethylene glycol and Cassia tora gum. Method for preparation of Levocetirizine Dihydrochloride is same as described in Example 2. Process for preparation of Colchicine films is same as described in example 1, except the final film forming mixture was warmed to 45 to 50° C. before casting. Nifedipine and Prednisolone films were formulated following the method of example 1. The prepared films were cut into units of 3.24 cm² and packed in individual pouches.

TABLE 15
Composition of oral quick disintegrating films containing a copolymer
of polyvinyl alcohol and polyethylene glycol and Cassiatora gum.

| | | | | mg/film* |
|---|---|---|---|---|
| | mg/film* | mg/film* | mg/film* | (% w/w |
| | % w/w of | (% w/w of | (% w/w of | of dry |
| Ingredients | dry film*) | dry film*) | dry film*) | film*) |
| Colchicine | — | 0.59 | — | — |
| | | (1.52) | | |
| Nifedipine | — | — | 9.96 | — |
| | | | (20.94) | |
| Levocetirizine | — | — | — | 1.25 |
| Dihydrochloride | | | | (3.16) |
| Prednisolone | 4.98 | — | — | — |
| | (11.45) | | | |
| A copolymer of | 35.39 | 35.39 | 34.39 | 34.39 |
| polyvinyl | (81.33) | (90.44) | (72.25) | (87.00) |
| alcohol and | | | | |
| polyethylene | | | | |
| glycol | | | | |
| Cassia toragum | 1.19 | 1.196 | 0.99 | 0.99 |
| | (2.74) | (3.05) | (2.09) | (2.52) |
| Aspartame | 0.94 | 0.94 | 1.14 | 1.14 |
| | (2.17) | (2.42) | (2.40) | (2.90) |
| Raspberry | 0.99 | 0.99 | 1.09 | 1.09 |
| flavor | (2.29) | (2.54) | (2.30) | (2.77) |
| Poloxamer188 | — | — | — | 0.64 |
| | | | | (1.63) |
| Distilled water | — | — | — | — |
| Total | 43.515 | 39.129 | 47.603 | 39.532 |
| | (100) | (100) | (100) | (100) |

Film Properties

| | | | | |
|---|---|---|---|---|
| Average | 36.62 ± 0.62 | 2.01 ± 0.43 | 14.28 ± 0.70 | 16.35 ± 1.05 |
| Disintegration | | | | |
| time (sec) | | | | |
| Average | 0.154 ± 0.01 | 0.103 ± 0.009 | 0.137 ± 0.014 | |
| Thickness (mm) | | | | |
| Average | 330 | — | 150 | |
| Dissolution time | | | | |
| (Time required | | | | |
| for >80% drug | | | | |
| release in | | | | |
| seconds) | | | | |
| Uniformity of | — | 99.57 ± 1.21 | | |
| drug content | | | | |

Note:
*Assuming all solvent evaporated

### Example 16

Oral quick disintegrating films were prepared from uniform polymeric, mixture of a copolymer of polyvinyl alcohol and polyethylene glycol and Guar gum. Method employed was same as described in Example 2 except that the final mixture was warmed to 45 to 50° C. before casting. The surface area of the final unit dose was 3.24 cm².

TABLE 16
Composition of oral quick disintegrating film containing a copolymer
of polyvinyl alcohol and polyethylene glycol and Guar gum.

| | | % w/w Dry |
|---|---|---|
| Ingredients | mg/film* | film* |
| Colchicine | 0.59 | 1.51 |
| A copolymer of | 35.39 | 89.64 |
| polyvinyl alcohol | | |
| and polyethylene | | |
| glycol | | |
| Guar gum | 0.89 | 2.27 |
| Aspartame | 0.99 | 1.52 |
| Raspberry flavor | 1.09 | 2.77 |
| Poloxamer188 | 0.49 | 1.26 |
| Distilled water | — | — |
| Total | 39.47 | 100 |

Note:
*Assuming all solvent evaporated

### Example 17

Process: The film forming ingredient a copolymer of polyvinyl alcohol and polyethylene glycol was dissolved in half the quantity of water. Gums: Xanthan gum, guar gum were soaked in this mixture for about one hour and stirred uniformly. Corn starch was slowly added under continuous mixing for two hours.

Atenolol was added in poloxamer solution separately prepared in remaining portion of water. Flavoring agent and sweetener were dissolved in the drug phase. The drug phase was slowly added to the polymer mixture and stirred uniformly. This final gel mixture was casted on a petri dish, dried in hot air oven and cut in desired dimension. The surface area of unit dose was 4.5 cm².

TABLE 17
Composition of oral quick disintegrating film
containing a copolymer of polyvinyl alcohol and
polyethylene glycol, Xanthan gum and Guar gum.

| | | % w/w |
|---|---|---|
| Ingredients | mg/film* | Dry film* |
| Atenolol | 9.96 | 16.82 |
| A copolymer of | 42.92 | 72.43 |
| polyvinyl | | |
| alcohol and | | |
| polyethylene | | |
| glycol | | |
| Xanthan Gum | 0.62 | 1.05 |
| Guar gum | 1.10 | 1.86 |
| Poloxamer 188 | 0.83 | 1.40 |
| Aspartame | 1.59 | 2.68 |
| Flavors | 1.38 | 2.33 |
| Corn starch | 0.83 | 1.40 |
| Distilled water | — | — |
| Total | 59.26 | 100 |

Film Properties

| | |
|---|---|
| Average Disintegration time (sec) | 22.15 ± 1.34 |
| Average Thickness (mm) | 0.118 ± 0.003 |

Note:
*Assuming all solvent evaporated.

### Example 18

Formulation procedure for preparation of oral quick disintegrating films containing a copolymer of polyvinyl alcohol and polyethylene glycol, Xanthan Gum, Locust Bean gum and Guar gum was same as described in Example 7. Octopamine HCl films were prepared with continuous warming at 45 to 50° C. of drug phase and polymer phase. The surface area of unit dose for Hydrochlorothiazide film was 4.5 cm², Lamotrigine 8 cm²and Octopamine HCl 8 cm².

TABLE 18
Composition of oral quick disintegrating films containing a copolymer
of polyvinyl alcohol and polyethylene glycol, Xanthan Gum, Locust
Bean gum and Guar gum

| | mg/film * | mg/film * | mg/film * |
|---|---|---|---|
| | (% w/w of | (% w/w of | (% w/w of |
| Ingredients | dry film *) | dry film *) | dry film *) |
| Lamotrigine | — | 24.98 | — |
| | | (20.0) | |
| Octopamine HCl | — | — | 25.10 |
| | | | (20.77) |
| Hydrochlorothiazide | 12.43 | — | — |
| | (11.30) | | |
| A copolymer of polyvinyl | 84.8 | 87.38 | 84.8 |
| alcohol and polyethylene | (77.09) | (70.30) | (70.16) |
| glycol | | | |
| Xanthan Gum | 0.73 | 0.49 | 0.86 |
| | (0.67) | (0.39) | (0.71) |
| Locust Bean gum | 1.84 | 2.21 | 1.72 |
| | (1.67) | (1.78) | (1.42) |
| Guar gum | 1.47 | 2.46 | 1.35 |
| | (1.33) | (1.98) | (1.12) |
| Poloxamer188 | 1.6 | 1.6 | 1.6 |
| | (1.45) | (1.28) | (1.32) |
| Aspartame | 2.70 | 2.83 | 2.95 |
| | (2.46) | (2.27) | (2.44) |
| Raspbery flavor | 2.21 | — | — |
| | (2.01) | | |
| Vanilla mint flavor | 2.46 | 2.33 | 2.46 |
| | (2.22) | (1.88) | (2.03) |
| Distilled water | — | — | — |
| Total | 110.27 | 124.3 | 120.86 |
| | (100) | (100) | (99.99) |

Film Properties

| | | | |
|---|---|---|---|
| Average Disintegration time | 31.24 ± 0.51 | 31.65 ± 0.78 | 22.05 ± 0.63 |
| (sec) | | | |
| Average Thickness (mm) | 0.072 ± 0.014 | 0.065 ± 0.005 | 0.071 ± 0.004 |

Note:
* Assuming all solvent evaporated

### Example 19

Procedure same as described in Example 7, with continuous warming of drug phase and polymer phase at 45 to 50° C. The surface area of unit dose was 4.5 cm².

TABLE 19
Composition of oral quick disintegrating film containing a copolymer
of polyvinyl alcohol and polyethylene glycol, Xanthan Gum and
Locust Bean gum

| | mg/film* | mg/film* | mg/film* |
|---|---|---|---|
| | (% w/w of | (% w/w of | (% w/w of |
| Ingredients | dry film*) | dry film*) | dry film*) |
| Octopamine HCl | 25.13 | 49.9 | 49.9 |
| | (31.67) | (45.2) | (45.04) |
| A copolymer of | 48.32 | 54 | 54 |
| polyvinyl alcohol | (60.90) | (48.9) | (48.65) |
| and polyethylene | | | |
| glycol | | | |
| Xanthan Gum | 0.27 | 0.34 | 0.27 |
| | (0.34) | (0.31) | (0.24) |
| Locust Bean gum | 0.96 | 0.96 | 0.969 |
| | (1.22) | (0.87) | (0.87) |
| Poloxamer 188 | 0.76 | 0.76 | 0.76 |
| | (0.95) | (0.69) | (0.68) |
| Aspartame | 1.66 | 1.8 | 1.8 |
| | (2.09) | (1.63) | (1.62) |
| Flavors | 1.38 | 1.45 | 1.45 |
| | (1.74) | (1.31) | (1.31) |
| Corn starch | 0.83 | — | — |
| | (1.04) | | |
| Hypromellose | — | — | 0.69 |
| | | | (0.62) |
| Guar gum | — | 1.03 | 1.03 |
| | | (0.94) | (0.93) |
| Distilled water | — | — | — |
| Total | 79.3 | 110.35 | 110.97 |
| | (100) | (100) | (100.56) |

Film Properties

| | | | |
|---|---|---|---|
| Average | 21.2 ± 0.19 | — | — |
| Disintegration time | | | |
| (sec) | | | |
| Average Thickness | 0.098 ± 0.007 | — | — |
| (mm) | | | |

Note:
*Assuming all solvent evaporated

Few films are tested for drug content on stability, data is as follows:

TABLE 20
Stability data for Amlodipine besylate (2.5 mg) films

| | Storage | | | | |
|---|---|---|---|---|---|
| Storage | duration | Visual | | | |
| condition | (weeks) | observation | Weight (mg) | Drug content | pH |
| Room | 0 | No change | 26.16 ± 0.75 | 96.82 ± 0.45 | 6-7 |
| Temperature | 2 | No change | 25.33 ± 1.03 | 99.99 ± 0.52 | 6-7 |
| | 4 | No change | 25.66 ± 0.81 | 98.68 ± 0.50 | 6-7 |
| | 8 | No change | 26.33 ± 0.81 | 100.82 ± 0.69 | 6-7 |
| 40° C., 75% | 0 | No change | 24.83 ± 0.98 | 99.50 ± 0.80 | 6-7 |
| RH | 2 | No change | 24.66 ± 0.81 | 98.1 ± 0.95 | 6-7 |
| | 4 | No change | 27 ± 0.89 | 97.93 ± 0.68 | 6-7 |
| | 8 | No change | 25.66 ± 1.03 | 98.38 ± 0.76 | 6-7 |

### Packaging of Oral Film

There are variety of options for packaging films of the present invention such as single pouch, blister card with multiple units, and continuous toll dispenser, depending on the application and marketing objectives. Final packaging must meet industry standards for patient compliance, such as labelling claims, instructions for use, child-resistant seals, and senior-friendly packaging. They must protect the film from environmental conditions. They must be FDA approved, by the drug regulatory/food regulatory authorities. They must be tamper-resistant, non-toxic, non-reactive with the product and must not impart to the product tastes or odors.

Single pouch: Peel-open laminates offer an ideal packaging solution for moisture sensitive medical and pharmaceutical products with high barrier properties. The peelable lamination seals and peels to itself, allowing for easy-opening, convenient product dispensing, portability and unit dosing. The pouch can be made transparent for product display.

Foil, paper or plastic pouches: The flexible pouch is a packaging concept capable of providing not only a package that is temper-resistance, but also by the proper selection of material, a package with a high degree of environmental protection. A flexible pouch is usually formed during the product filling operation by either vertical or horizontal forming, filling, or sealing equipment. The pouches can be single pouches or aluminium pouches.

Blister card with multiple units: The blister container consists of two components: the blister, which is the formed cavity that holds the product, and the lid stock, which is the material that seals to the blister. The film selection should be based upon the degree of protection required. Generally, the lid stock is made of aluminium foil. The material used to form the cavity is typically a plastic, which can be designed to protect the dosage form from moisture.

For branding purposes, converters may choose to print information directly onto the film unit doses before packaging. Criteria taken into consideration include the need for unit-dose packaging, barcode labelling and the content in instructions for use, child-resistant seals.

We claim:

1. An oral film, comprising: a medicinal or non-medicinal active, copolymer of polyvinyl alcohol and polyethylene glycol and at least one gum.

2. The oral film according to claim 1, wherein the copolymer of polyvinyl alcohol and polyethylene glycol is from 10% to 95%.

3. The oral film according to claim 1, wherein the gum is selected from a group consisting of Cassia tora, xanthan gum, guar gum, gum ghatti, veegum, locust bean gum, karaya gum and combinations thereof.

4. The oral film according to claim 1, wherein the gum is from 0.01% to 15%.

5. The oral film according to claim 1, further comprising one or more additional excipient selected from additional film forming agents, solubilizing agents, plasticizers, surfactants, binders, thickeners, stabilizers, disintegrants, fillers, sweeteners, saliva stimulating agents, flavouring agents, mouth freshening/breath freshening agents, cooling agents, gelling agents, colouring agents, effervescence agents and mucoadhesive agent.

6. The oral film according to claim 5, wherein the solubilizing agent or surfactant is poloxamer.

7. The oral film according to claim disintegrating within two minutes and preferably within a minute.

8. A process of preparing an oral film, comprising medicinal or non-medicinal active, comprising following steps:
a. Adding a copolymer of polyvinyl alcohol and polyethylene glycol to one part of a total quantity of a purified water under continuous stirring;
b. Adding a gum under stirring to above mixture;
c. Optionally adding any additional excipient and stirring until complete solubilization;
d. Adding the active agent to this mixture either in a form of solid or solution of active agent in cases where the active agent is insoluble;
e. Adding the rest of of water;
f. Casting said mixture on a casting surface and drying for more than 5 hrs;
g. Optionally cutting the film in desired dimension,
h. Optionally packing the films.

9. A process of preparing an oral film, comprising medicinal or non-medicinal active, comprising following steps:
a. Adding a copolymer of polyvinyl alcohol and polyethylene glycol to one part of a total quantity of a purified water under continuous stirring;
b. Soaking a gum in above solution for some time;
c. Optionally adding any additional excipient and stirring until complete solubilization;
d. Adding the active agent to this mixture either in a form of solid or solution of active agent in cases where the active agent is insoluble;
e. Adding the other part of the total quantity of water;
f. Casting said mixture on a casting surface and drying;
g. Optionally cutting the film in desired dimension,
h. Optionally packing the films.

10. The oral film according to claim 1 for oral, mucosal, sublingual administration of medicinal or non-medicinal active.
